(19) United States
(12) Patent Application Publication
(10) Pub. No.: US 2010/0113565 A1
(43) Pub. Date: May 6, 2010

(54) IMMUNOSTIMULATORY COMBINATIONS AND METHODS

(76) Inventors: Keith B. Gorden, Maplewood, MN (US); Qiu Xiaohong, Rosemount, MN (US); Paul D. Wightman, Woodbury, MN (US)

Correspondence Address: 3M INNOVATIVE PROPERTIES COMPANY, PO BOX 33427, ST. PAUL, MN, 55133-3427, US

(21) Appl. No.: 11/720,872

(22) PCT Filed: Dec. 8, 2005
(86) PCT No.: PCT/US05/44448

§ 371 Date: Jun. 5, 2007

Related U.S. Application Data
(60) Provisional application No. 60/634,146, filed on Dec. 8, 2004.

(51) Int. Cl. A61K 31/7088; C12N 15/87

(52) U.S. Cl. 514/44 R; 435/455

(57) ABSTRACT

The invention provides immunostimulatory compositions and methods of enhancing TLR8-mediated biological activity. Generally, the immunostimulatory compositions include a TLR8 agonist and an immunostimulatory oligonucleotide in an amount effective to enhance TLR8-mediated biological activity. The invention also provides methods of inducing TLR8-mediated biological activity in immune cells. Generally, the methods include contacting immune cells with an immunostimulatory composition that includes a TLR8 agonist and an immunostimulatory oligonucleotide in an amount effective to enhance TLR8-mediated biological activity. In some cases, the immunostimulatory composition provides a synergistic enhancement of TLR8-mediated biological activity.

### BACKGROUND

There has been a major effort in recent years, with significant success, to discover new drug compounds that act by stimulating certain key aspects of the immune system, as well as by suppressing certain other aspects (see, e.g., U.S. Pat. Nos. 6,039,969 and 6,200,592). These compounds, referred to herein as immune response modifiers (IRMs), appear to act through basic immune system mechanisms known as Toll-like receptors (TLRs) to induce selected cytokine biosynthesis, induction of co-stimulatory molecules, and increased antigen-presenting capacity. They may be useful for treating a wide variety of diseases and conditions. For example, certain IRMs may be useful for treating viral diseases (e.g., human papilloma virus, hepatitis, herpes), neoplasias (e.g., basal cell carcinoma, squamous cell carcinoma, actinic keratosis, melanoma), and T_{H}2-mediated diseases (e.g., asthma, allergic rhinitis, atopic dermatitis), autoimmune diseases (e.g., multiple sclerosis), and are also useful as vaccine adjuvants.

Many of the IRM compounds are small organic molecule imidazoquinoline amine derivatives (see, e.g., U.S. Pat. No. 4,689,338), but a number of other compound classes are known as well (see, e.g., U.S. Pat. Nos. 5,446,153; 6,194,425; and 6,110,929; and International Publication Number WO 2005/079195) and more are still being discovered.

Certain small molecule IRMs (smIRMs) possess potent immunomodulating activity such as, for example, antiviral and antitumor activity. Certain smIRMs modulate the production and secretion of cytokines. For example, certain smIRM compounds induce the production and secretion of cytokines such as, e.g., Type I interferons, TNF-α, IL-1, IL-6, IL-8, IL-10, IL-12, MIP-1, and/or MCP-1. As another example, certain smIRM compounds can inhibit production and secretion of certain T_{H}2 cytokines, such as IL-4 and IL-5. Additionally, some smIRM compounds are said to suppress IL-1 and TNF (U.S. Pat. No. 6,518,265).

Other IRMs have higher molecular weights, such as, for example, oligonucleotides, including CpG oligodinucleotides (ODNs, see, e.g., U.S. Pat. No. 6,194,388). At least three structurally distinct classes of synthetic CpG ODNs have been described. CpG-B ODNs (also referred to as K-type CpG ODNs) can trigger the differentiation of antigen presenting cells (APCs) and the proliferation of B cells. CpG-A ODNs (also referred to as D-type CpG ODNs) can directly induce the secretion of interferon-α (IFN-α) from plasmacytoid dendritic cells (pDCs), which indirectly supports the subsequent maturation of APCs. CpG-C ODNs can stimulate B cells to secrete interleukin-6 (IL-6) and pDCs to produce IFN-α, thereby combining some of the stimulatory properties of CpG-A ODNs and CpG-B ODNs.

In view of the great therapeutic potential for IRMs, and despite the important work that has already been done, there is a substantial ongoing need to expand their uses and therapeutic benefits.

### SUMMARY

It has been found that certain oligonucleotide sequences can enhance certain immunostimulatory activities of certain IRM compounds.

Accordingly, the present invention provides an immunostimulatory combination that generally includes a TLR8 agonist and an immunostimulatory oligonucleotide.

In another aspect, the present invention also provides a method of inducing TLR8-mediated biological activity in immune cells. Generally, the method includes contacting the immune cells with an immunostimulatory combination that includes a TLR8 agonist and an immunostimulatory oligonucleotide in an amount effective to increase a TLR8-mediated biological activity of the cells to a greater extent than contacting the immune cells with the TLR8 agonist without the immunostimulatory oligonucleotide.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1 shows enhancement of IRM-induced TLR8-mediated biological activity by CpG ODN immunostimulatory oligonucleotides in a transfected cell line.

FIG. 2 shows enhancement of IRM-induced TLR8-mediated biological activity by CpG ODN immunostimulatory oligonucleotides in a transfected cell line.

FIG. 3 shows enhancement of IRM-induced TLR8-mediated biological activity by CpG ODN immunostimulatory oligonucleotides in peripheral blood mononuclear cells (PBMCs).

FIG. 4 shows enhancement of IRM-induced TLR8-mediated biological activity by CpG ODN immunostimulatory oligonucleotides in peripheral blood mononuclear cells (PBMCs).

FIG. 5 shows enhancement of IRM-induced TLR8-mediated biological activity by CpG ODN immunostimulatory oligonucleotides in monocyte-derived dendritic cells.

FIG. 6 shows enhancement of IRM-induced TLR8-mediated biological activity by CpG ODN immunostimulatory oligonucleotides in monocyte-derived dendritic cells.

FIG. 7 shows enhancement of IRM-induced TLR8-mediated biological activity by poly(A) immunostimulatory oligonucleotides of varying lengths in a transfected cell line.

FIG. 8 shows enhancement of IRM-induced TLR8-mediated biological activity by poly(C) immunostimulatory oligonucleotides of varying lengths in PBMCs.

FIG. 9 shows enhancement of IRM-induced TLR8-mediated biological activity by poly(T) immunostimulatory oligonucleotides of varying lengths in PBMCs.

### DETAILED DESCRIPTION OF ILLUSTRATIVE EMBODIMENTS OF THE INVENTION

The present invention exploits the observation that certain oligonucleotide sequences can enhance induction of certain TLR8-mediated biological activities in a dose dependent manner.

In one aspect, the invention provides immunostimulatory combinations that include a TLR8 agonist and an immunostimulatory oligonucleotide. Each component may, by itself, possess a certain immunostimulatory activity. In many cases, the combination of a TLR8 agonist and an immunostimulatory oligonucleotide can provide greater immunostimulatory activity than either component can provide alone. In some cases, the combination of a TLR8 agonist and an immunostimulatory oligonucleotide can provide, for example, a two-fold, three-fold, five-fold, or even greater increase in at least one TLR8-mediated biological activity compared to that induced by a TLR8 agonist administered without the immunostimulatory oligonucleotide. In certain cases, the combination of components can provide synergistic immunostimulatory activity.

In another aspect, the invention provides a method of enhancing induction of TLR8-mediated biological activity of immune cells. In practice, the method may be used, for example, to improve the efficacy of certain immunological treatments that involve a TLR8-mediated biological activity. Such treatments can include, for example, a therapeutic or prophylactic vaccine. Thus, for example, the invention may enhance vaccine-induced TLR8-mediated biological activity sufficiently to improve the efficacy of the vaccine—even to the point of enabling a vaccine previously considered ineffective to be considered effective.

Alternatively, the invention may permit effective treatment of a condition using less of a component of an immunological composition (e.g., the antigen or an adjuvant of a vaccine). This may be desirable if a particular component, while useful for generating a desired immunological response, is expensive, difficult to obtain, or generates undesirable side effects. Thus, the invention may enable some immunological treatments to be clinically and/or commercially viable that previously had been considered clinically and/or commercially undesirable because of, for example, (a) cost of a component of the treatment, (b) availability of all components, and/or (c) the amount of a component (e.g., an antigen) previously considered necessary to generate an effective immune response also generated an undesirable level of side effects.

For purposes of this invention, the following terms shall have the meanings set forth as follows:

“Agonist” refers to a compound that can combine with a receptor (e.g., a TLR) to induce a biological activity. An agonist may be a ligand that directly binds to the receptor. Alternatively, an agonist may combine with a receptor indirectly by, for example, (a) forming a complex with another molecule that directly binds to the receptor, or (b) otherwise results in the modification of another compound so that the other compound directly binds to the receptor (e.g., cellular signaling). An agonist may be referred to as an agonist of a particular TLR (e.g., a TLR8 agonist) or a particular combination of TLRs (e.g., a TLR 7/8 agonist—an agonist of both TLR7 and TLR8).

“Agonist-receptor interaction” refers to any direct or indirect interaction such as, for example, binding, forming a complex, or biochemical modification that induces a cellular activity.

“Immune cell” refers to a cell of the immune system, i.e., a cell directly or indirectly involved in the generation or maintenance of an immune response, regardless of whether the immune response is innate or acquired, humoral or cell-mediated.

“Immunostimulatory oligonucleotide” refers to an oligonucleotide sequence that is capable of measurably enhancing TLR8-mediated biological activity.

“Induce” and variations thereof refer to any measurable increase in biological activity. For example, induction of a particular cytokine refers to an increase in the production of the cytokine.

“Inhibit” and variations thereof refer to any measurable reduction of biological activity. For example, inhibition of a particular cytokine refers to a decrease in production of the cytokine. The extent of inhibition may be characterized as a percentage of a normal level of activity.

“IRM compound” refers generally to a compound that alters the level of one or more immune regulatory molecules, e.g., cytokines or co-stimulatory markers, when administered to an IRM-responsive cell. Representative IRM compounds include the small organic molecules, purine derivatives, small heterocyclic compounds, amide derivatives, and oligonucleotide sequences described below.

“Selective” and variations thereof refer to having a differential impact on biological activity to any degree. An agonist that selectively modulates biological activity through a particular TLR may be a TLR-selective agonist. TLR-selectivity may be described with respect to a particular TLR (e.g., TLR8-selective) or with respect to a particular combination of TLRs (e.g., TLR 7/9-selective). A TLR selective (e.g., TLR8-selective) compound may exclusively induce biological activity mediated by the indicated TLR (i.e., TLR-specific), or may induce biological activity mediated through multiple TLRs, but induce activity mediated through the indicated TLR to a greater extent than any other TLR (i.e., TLR-dominant such as, for example, TLR8-dominant).

“smIRM” refers generally to a small molecule IRM compound, an IRM compound having a molecular weight of about 1 kilodalton (kDa) or less.

“Synergistic” and variations thereof refer to an interaction of a TLR8 agonist and an immunostimulatory oligonucleotide so that their combined immunological effect is greater than the sum of their individual effects.

“TLR-mediated” refers to a biological activity (e.g., cytokine production) that results, directly or indirectly, from TLR function. A particular biological activity may be referred to as mediated by a particular TLR (e.g., “TLR8-mediated”).

Also herein, the recitations of numerical ranges by endpoints include all numbers subsumed within that range (e.g., 1 to 5 includes 1, 1.5, 2, 2.75, 3, 3.80, 4, 5, etc.).

The TLR agonism of a particular compound may be assessed in any suitable manner. For example, assays and recombinant cell lines suitable for detecting TLR agonism of test compounds are described, for example, in U.S. Patent Publication Nos. US2004/0014779, US2004/0132079, US2004/0162309, US2004/0171086, US2004/0191833, and US2004/0197865.

Regardless of the particular assay employed, a compound can be identified as an agonist of a particular TLR if performing the assay with a compound results in at least a threshold increase of some biological activity mediated by the particular TLR. Conversely, a compound may be identified as not acting as an agonist of a specified TLR if, when used to perform an assay designed to detect biological activity mediated by the specified TLR, the compound fails to elicit a threshold increase in the biological activity. Unless otherwise indicated, an increase in biological activity refers to an increase in the same biological activity over that observed in an appropriate control. An assay may or may not be performed in conjunction with the appropriate control. With experience, one skilled in the art may develop sufficient familiarity with a particular assay (e.g., the range of values observed in an appropriate control under specific assay conditions) that performing a control may not always be necessary to determine the TLR agonism of a compound in a particular assay.

The precise threshold increase of TLR-mediated biological activity for determining whether a particular compound is or is not an agonist of a particular TLR in a given assay may vary according to factors known in the art including but not limited to the biological activity observed as the endpoint of the assay, the method used to measure or detect the endpoint of the assay, the signal-to-noise ratio of the assay, the precision of the assay, and whether the same assay is being used to determine the agonism of a compound for multiple TLRs. Accordingly it is not practical to set forth generally the threshold increase of TLR-mediated biological activity required to identify a compound as being an agonist or a non-agonist of a particular TLR for all possible assays. Those of ordinary skill in the art, however, can readily determine the appropriate threshold with due consideration of such factors.

Assays employing HEK293 cells transfected with an expressible TLR structural gene may use a threshold of, for example, at least a three-fold increase in a TLR-mediated biological activity (e.g., NF-κB activation) when the compound is provided at a concentration of, for example, from about 1 μM to about 10 μM for identifying a compound as an agonist of the TLR transfected into the cell. However, different thresholds and/or different concentration ranges may be suitable in certain circumstances. Also, different thresholds may be appropriate for different assays.

In one aspect, the invention provides a method of enhancing TLR8-mediated biological activity of immune cells. In some cases, for example, a TLR8 agonist may be an agonist of at least one additional TLR (e.g., TLR7, a so-called TLR7/8 agonist) and may, therefore, ordinarily induce TLR8-mediated biological activity as well as biological activity mediated by one or more additional TLRs (e.g., TLR7-mediated biological activity). Practicing the invention may be used to enhance the TLR8-mediated biological activity and in some cases limit—or even eliminate—biological activity induced by the compound that is mediated by another (e.g., non-TLR8) TLR.

Thus, the method may be used to enhance the TLR8-mediated biological activity so that a compound possessing mixed TLR agonism acts more like a TLR-selective compound. In some cases, the compound may act essentially as a TLR8-dominant compound. In certain cases, the method may further decrease the extent to which the compound induces biological activity mediated by another TLR so that the compound acts essentially as a TLR8-specific compound. For example, reducing—or even eliminating—the TLR7-mediated biological activity of a TLR7/8 agonist may make the compound act essentially as a TLR8-selective agonist (e.g., as a TLR8-dominant agonist or a TLR8-specific agonist).

As an example, one TLR8-mediated biological activity can include production of tumor necrosis factor (TNF), which may be beneficial for treating certain conditions such as, for example, certain cancers (e.g., melanoma). On the other hand, TLR7-mediated biological activity can include production of interferon-α (IFN-α), which may aggravate certain conditions such as, for example, lupus erythematosus. A particular TLR7/8 agonist may be identified as being well-suited for treating certain cancers such as, for example, melanoma, perhaps because of efficacy and/or the extent of TLR8-mediated biological activity induced by the compound, but also perhaps because of other desirable characteristics such as, for example, low toxicity, being easy to formulate and deliver (formulability), cost, stability (e.g., shelf-life), bio-availability, metabolic half-life, etc. However, if administered to a subject having lupus erythematosus, the TLR7-mediated biological activity (IFN-α production) induced by the compound may aggravate the lupus erythematosus to an extent that may prevent consideration of the TLR7/8 compound as a treatment for cancer in a patient that has been diagnosed with lupus erythematosus.

Practicing the present invention may allow such a subject to enjoy the benefits of treating one condition (e.g., the cancer) with the TLR7/8 compound without aggravating the second condition (e.g., lupus erythematosus) to an intolerable extent. By administering a sufficient amount of an immunostimulatory oligonucleotide with the TLR7/8 agonist, sufficient TLR8-mediated biological activity may be induced by the TLR7/8 compound to provide treatment for the cancer, while the TLR7-mediated biological activity induced by the TLR7/8 compound may be reduced to acceptable levels—in some cases, even fully eliminating the TLR7-mediated biological activity. Thus, in the example above, administering the combination of the TLR7/8 agonist and immunostimulatory oligonucleotide may induce sufficient TNF to treat the cancer and reduce the amount of IFN-α induced by the TLR7/8 agonist sufficiently so that the treatment of the cancer may proceed while limiting—or even eliminating—aggravation of the lupus erythematosus that would otherwise result from administering the TLR7/8 agonist.

In another aspect, the invention provides immunostimulatory combinations that are effective for enhancing TLR8-mediated biological activity. In some cases, the combination can include a TLR8 agonist and an immunostimulatory oligonucleotide in an amount effective to increase the extent to which the TLR8 agonist induces at least one TLR8-mediated biological activity. The TLR8 agonist and the immunostimulatory oligonucleotide may exist in a single formulation or, alternatively, the two components may exist in separate formulations. Formulations suitable for use in practicing the invention are described in detail below.

Exemplary TLR8-mediated biological activities that may be modulated while practicing the invention can include, for example, induction of co-stimulatory marker expression (e.g., CD40, CD80, CD86, etc.), induction of surface marker expression (e.g., CCR7), activation of NF-κB, induction of an intercellular adhesion molecule (ICAM, e.g., ICAM-1, ICAM-2, I-CAM-3, etc.), increased antigen-presenting capability, maturation of plasmacytoid dendritic cells (pDCs), proliferation of B lymphocytes, and induction of certain cytokines. Cytokine induced by a TLR8-mediated biological activity include, for example, TNF-α, a Type I interferon (e.g., IFN-α, IFN-β, IFN-ω, etc.), IFN-γ, IL-1, IL-6, IL-8, IL-10, IL-12, MIP-1, MCP-1, or any combination thereof.

The TLR8 agonist may be any compound capable, or potentially capable when administered in combination with an immunostimulatory oligonucleotide, of inducing at least one TLR8-mediated biological activity. In some cases, the TLR8 agonist may be an IRM compound. Suitable IRM compounds are described in detail below.

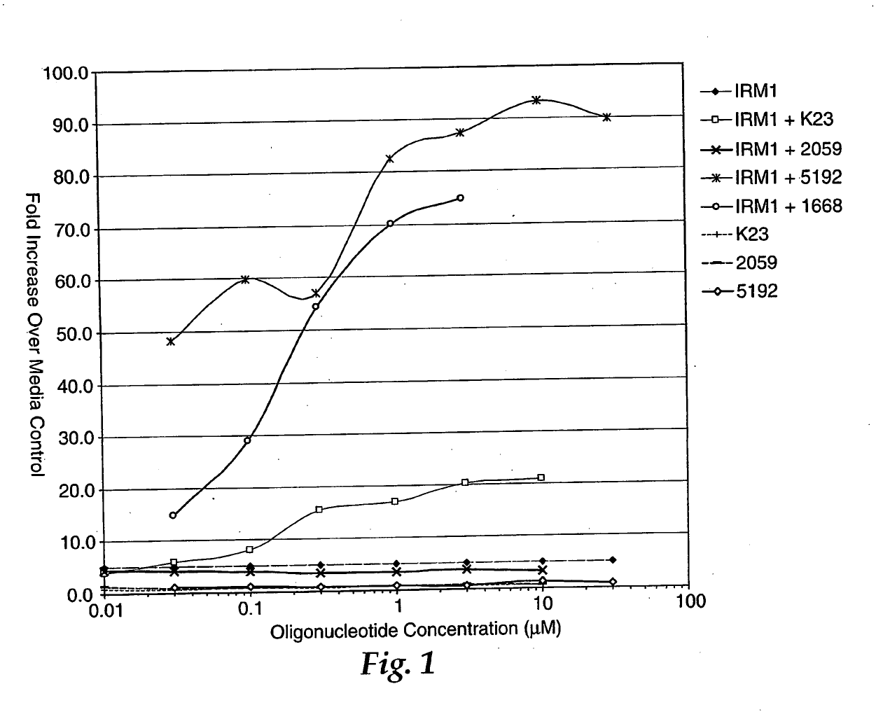

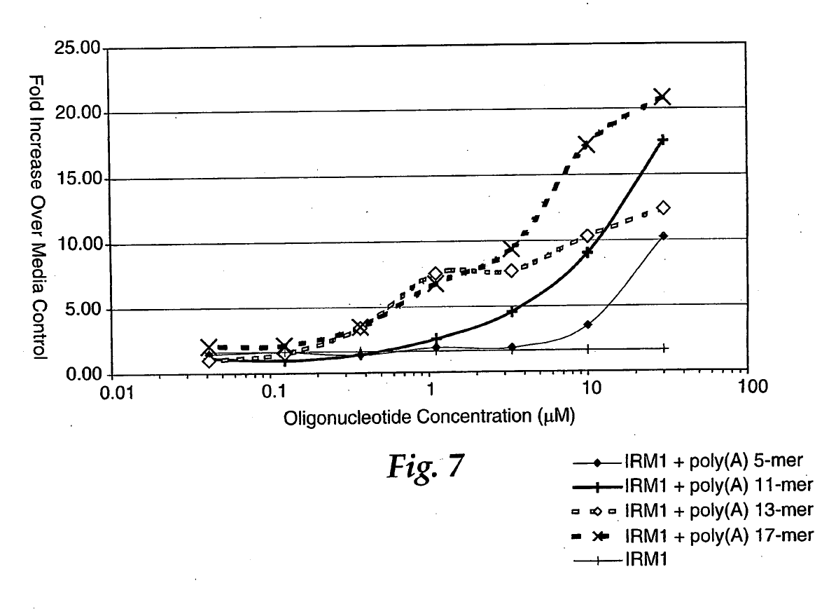

The immunostimulatory oligonucleotide may be any suitable oligonucleotide sequence—i.e., an oligonucleotide sequence capable of enhancing at least one TLR8-mediated biological activity induced by a TLR8 agonist. In some embodiments, a suitable immunostimulatory oligonucleotide may contain CpG ODN sequences such as, for example, CpG-A ODN, CpG-B ODN, or CpG-C ODN sequences (FIGS. 1-6). However, other oligonucleotide sequences may be suitable as well. For example, poly(A), poly(C) and poly(T) oligonucleotides have been identified as being capable of enhancing TLR8-mediated biological activity (FIGS. 7-9).

In some embodiments, the immunostimulatory oligonucleotide can have a stacked secondary structure that may permit certain compounds (e.g., certain IRM compounds) to intercalate into the oligonucleotide sequence. Intercalation of a compound into the oligonucleotide may result in the formation of a complex that preferentially interacts with TLR8. Thus certain compounds that would ordinarily not possess measurable TLR8 agonism may, when complexed with an immunostimulatory oligonucleotide, act as TLR8 agonists. Also, compounds that would ordinarily possess mixed TLR agonism may, when complexed with an immunostimulatory oligonucleotide, act more like TLR8-selective agonists.

Certain IRMs are small organic molecules (smIRMs, e.g., molecular weight under about 1000 Daltons, in some cases under about 500 Daltons, as opposed to large biological molecules such as proteins, peptides, and the like) such as those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; 6,797,718; and 6,818,650; U.S. Patent Publication Nos. 2004/0091491; 2004/0147543; and 2004/0176367; and International Publication Nos. WO 2005/18551, WO 2005/18556, WO 2005/20999, WO 2005/032484, WO 2005/048933, WO 2005/048945, WO 2005/051317, WO 2005/051324, WO 2005/066169, WO 2005/066170, WO 2005/066172, WO 2005/076783, and WO 2005/079195.

Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U. S. Pat. Nos. 6,376,501; 6,028,076 and 6,329,381; and in WO 02/08905), and certain 3-β-D-ribofuranosylthiazolo[4,5-d]pyrimidine derivatives (such as those described in U.S. Publication No. 2003/0199461).

Other IRMs include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunostimulatory structural motifs such as those described, for example, in U.S. Pat. Nos. 6,426,334 and 6,476,000. Other IRM nucleotide sequences lack CpG sequences and are described, for example, in International Patent Publication No. WO 00/75304.

Other IRMs include biological molecules such as aminoalkyl glucosaminide phosphates (AGPs) and are described, for example, in U.S. Pat. Nos. 6,113,918; 6,303,347; 6,525,028; and 6,649,172.

Unless otherwise indicated, reference to a compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

In some embodiments of the present invention, the IRM compound may include a 2-aminopyridine fused to a five membered nitrogen-containing heterocyclic ring, or a 4-aminopyrimidine fused to a five membered nitrogen-containing heterocyclic ring.

IRM compounds suitable for use in the invention include compounds having a 2-aminopyridine fused to a five membered nitrogen-containing heterocyclic ring. Such compounds include, for example, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, hydroxylamine substituted imidazoquinoline amines, oxime substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, hydroxylamine substituted tetrahydroimidazoquinoline amines, oxime substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; pyrazolopyridine amines; pyrazoloquinoline amines; tetrahydropyrazoloquinoline amines; pyrazolonaphthyridine amines; tetrahydropyrazolonaphthyridine amines; and 1H-imidazo dimers fused to pyridine amines, quinoline amines, tetrahydroquinoline amines, naphthyridine amines, or tetrahydronaphthyridine amines.

In certain embodiments, the IRM compound may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

In certain other embodiments, the IRM compound may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

As used herein, a substituted imidazoquinoline amine refers to an amide substituted imidazoquinoline amine, a sulfonamide substituted imidazoquinoline amine, a urea substituted imidazoquinoline amine, an aryl ether substituted imidazoquinoline amine, a heterocyclic ether substituted imidazoquinoline amine, an amido ether substituted imidazoquinoline amine, a sulfonamido ether substituted imidazoquinoline amine, a urea substituted imidazoquinoline ether, a thioether substituted imidazoquinoline amine, a hydroxylamine substituted imidazoquinoline amine, an oxime substituted imidazoquinoline amine, a 6-, 7-, 8-, or 9-aryl, heteroaryl, aryloxy or arylalkyleneoxy substituted imidazoquinoline amine, or an imidazoquinoline diamine. As used herein, substituted imidazoquinoline amines specifically and expressly exclude 1-(2-methylpropyl)-1H-imidazo[4,5-c]quinolin-4-amine and 4-amino-α,α-dimethyl-2-ethoxymethyl-1H-imidazo[4,5-c]quinolin-1-ethanol.

In certain embodiments, the IRM compound may be a thiazoloquinoline amine such as, for example, 2-propylthiazolo[4,5-c]quinolin-4-amine or N43-(4-amino-2-propylthiazolo[4,5-c]quinolin-7-yl)phenyl]methanesulfonamide.

Suitable IRM compounds also may include the purine derivatives, imidazoquinoline amide derivatives, benzimidazole derivatives, adenine derivatives, aminoalkyl glucosaminide phosphates, and oligonucleotide sequences described above.

An immunostimulatory combination may be provided in a single formulation that includes an immunostimulatory oligonucleotide. In other cases, an immunostimulatory combination may include an immunostimulatory oligonucleotide and an IRM compound. Alternatively, an immunostimulatory combination may include a plurality of formulations in which the IRM compound and the immunostimulatory oligonucleotide may be provided in the same formulation or in different formulations. Formulations suitable for use in connection with therapeutic combinations of the invention are described in detail below.

An immunostimulatory combination may be provided in any formulation or combination of formulations suitable for administration to a subject. Suitable types of formulations are described, for example, in U.S. Pat. No. 5,736,553; U.S. Pat. No. 5,238,944; U.S. Pat. No. 5,939,090; U.S. Pat. No. 6,365,166; U.S. Pat. No. 6,245,776; U.S. Pat. No. 6,486,186; European Patent No. EP 0 394 026; and International Patent Publication No. WO 03/045391. A formulation may be provided in any suitable form including, but not limited to, a solution, a suspension, an emulsion, or any form of mixture. A formulation may include any pharmaceutically acceptable excipient, carrier, or vehicle. For example, a formulation may be delivered in a conventional dosage form such as, for example, a cream, an ointment, an aerosol formulation, a non-aerosol spray, a gel, a lotion, a tablet, an elixir, and the like. A formulation may further include one or more additives including but not limited to adjuvants, skin penetration enhancers, colorants, flavorings, fragrances, moisturizers, thickeners, and the like.

A formulation may be administered in any suitable manner such as, for example, non-parenterally or parenterally. As used herein, non-parenterally refers to administration through the digestive tract, including by oral ingestion. Parenterally refers to administration other than through the digestive tract such as, for example, intravenously, intramuscularly, transdermally, subcutaneously, transmucosally (e.g., by inhalation), or topically.

The composition of a formulation suitable for practicing the invention may vary according to factors known in the art including but not limited to the physical and chemical nature of the immunostimulatory oligonucleotide, the nature of the carrier, the intended dosing regimen, the state of the subject's immune system (e.g., suppressed, compromised, stimulated), the method of administering the immunostimulatory oligonucleotide, the nature and potency of any TLR8 agonist administered with the immunostimulatory oligonucleotide (if any), and the species to which the formulation is being administered. Accordingly, it is not practical to set forth generally the composition of a formulation effective for all possible applications. Those of ordinary skill in the art, however, can readily determine an appropriate formulation with due consideration of such factors.

In some embodiments, a suitable formulation may include, for example, from about 0.0001% to about 10% immunomodulatory oligonucleotide, although in some embodiments the formulation may include immunomodulatory oligonucleotide in a concentration outside of this range. For example, a formulation may include from about 0.01% to about 1% immunomodulatory oligonucleotide.

In some embodiments, the methods of the present invention may include administering IRM to a subject in a formulation of, for example, from about 0.0001% to about 10% to the subject, although in some embodiments the IRM compound may be administered using a formulation that provides IRM compound in a concentration outside of this range. In certain embodiments, the method includes administering to a subject a formulation that includes from about 0.01% to about 5% IRM compound, for example, a formulation that includes from about 0.1% to about 0.5% IRM compound.

An amount of an immunostimulatory oligonucleotide effective for enhancing TLR8-mediated biological activity of immune cells is an amount sufficient to increase at least one TLR8-mediated biological activity. The precise amount of immunostimulatory oligonucleotide required to be effective may vary according to factors known in the art such as, for example, the physical and chemical nature of the immunostimulatory oligonucleotide, the nature of the carrier, the intended dosing regimen, the state of the subject's immune system (e.g., suppressed, compromised, stimulated), the method of administering the immunostimulatory oligonucleotide, the potency of the TLR8 agonist being administered with the immunostimulatory oligonucleotide, and the species to which the formulation is being administered. Accordingly, it is not practical to set forth generally the amount that constitutes an amount of immunostimulatory oligonucleotide effective for all possible applications. Those of ordinary skill in the art, however, can readily determine the appropriate amount with due consideration of such factors.

In some embodiments, the methods of the present invention include administering sufficient immunostimulatory oligonucleotide to provide a dose of, for example, from about 100 ng/kg to about 50 mg/kg to the subject, although in some embodiments the methods may be performed by administering immunostimulatory oligonucleotide in a dose outside this range. In some of these embodiments, the method includes administering sufficient immunostimulatory oligonucleotide to provide a dose of from about 10 μg/kg to about 5 mg/kg to the subject, for example, a dose of from about 100 μg/kg to about 1 mg/kg.

An amount of TLR8 agonist that is effective for practicing the invention is an amount that, in combination with an immunostimulatory oligonucleotide, is capable of inducing at least one TLR8-mediated biological activity. Thus, in some cases, a TLR8 agonist may be provided in an amount that, if administered without the immunostimulatory oligonucleotide, ordinarily may not induce TLR8-mediated biological activity, but is capable of inducing TLR8-medfiated biological activity when provided with the immunostimulatory oligonucleotide.

In some embodiments, the methods of the present invention include administering sufficient TLR8 agonist to provide a dose of, for example, from about 100 ng/kg to about 50 mg/kg to the subject, although in some embodiments the methods may be performed by administering the TLR8 agonist in a dose outside this range. In some of these embodiments, the method includes administering sufficient TLR8 agonist to provide a dose of from about 10 μg/kg to about 5 mg/kg to the subject, for example, a dose of from about 100 μg/kg to about 1 mg/kg.

The dosing regimen may depend at least in part on many factors known in the art including but not limited to the physical and chemical nature of the immunostimulatory oligonucleotide, the nature of the carrier, the amount of immunostimulatory oligonucleotide being administered, the state of the subject's immune system (e.g., suppressed, compromised, stimulated), the method of administering the immunostimulatory oligonucleotide, the desired result, and the potency of the TLR8 agonist being administered with the immunostimulatory oligonucleotide, and the species to which the formulation is being administered. Accordingly it is not practical to set forth generally the dosing regimen effective for all possible applications. Those of ordinary skill in the art, however, can readily determine an appropriate dosing regimen with due consideration of such factors.

In some embodiments, the immunostimulatory combination may be administered on an “as needed” basis, i.e., whenever symptoms or conditions arise for which administration of the combination is desired. In some cases, the immunostimulatory combination may be administered only once. In other embodiments, the immunostimulatory combination may be administered at a frequency of, for example, from about once per day to about once per month, although in some embodiments the methods may be performed by administering the immunostimulatory combination at a frequency outside this range.

Conditions that may be treated by practicing the invention include, but are not limited to:

(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);

(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;

(c) other infectious diseases, such chlamydia, fungal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection; and

(d) neoplastic diseases, such as intraepithelial neoplasias, cervical dysplasia, actinic keratosis, basal cell carcinoma, squamous cell carcinoma, Kaposi's sarcoma, melanoma, renal cell carcinoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers;

(e) T_{H}2-mediated, atopic diseases, such as atopic dermatitis or eczema, eosinophilia, asthma, allergy, allergic rhinitis, and Ommen's syndrome;

(f) certain autoimmune diseases such as systemic lupus erythematosus, essential thrombocythaemia, multiple sclerosis, discoid lupus, alopecia areata; and

(g) diseases associated with wound repair such as, for example, inhibition of keloid formation and other types of scarring (e.g., enhancing wound healing, including chronic wounds).

Additionally, an immunostimulatory combination may be useful as a vaccine adjuvant for use in conjunction with any material that raises either humoral and/or cell mediated immune response, such as, for example, live viral, bacterial, or parasitic immunogens; inactivated viral, tumor-derived, protozoal, organism-derived, fungal, or bacterial immunogens, toxoids, toxins; self-antigens; polysaccharides; proteins; glycoproteins; peptides; cellular vaccines; DNA vaccines; autologous vaccines; recombinant proteins; glycoproteins; peptides; and the like, for use in connection with, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, yellow fever, and Alzheimer's Disease.

The methods of the present invention may be performed on any suitable subject. Suitable subjects include but are not limited to animals such as but not limited to humans, non-human primates, rodents, dogs, cats, horses, pigs, sheep, goats, or cows.

### Examples

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

The IRM compounds used in the examples are shown in Table 1. The immunostimulatory oligonucleotides used in the examples are shown in Table 2.

TABLE 1

| Compound | Chemical Name | Reference |
|---|---|---|
| IRM1 | 2-propylthiazolo[4,5-c]quinolin-4-amine | U.S. Pat. No. |
| | | 6,110,929 |
| | | Example 12 |
| IRM2 | N-[3-(4-amino-2-propylthiazolo[4,5- | U.S. Ser. No. |
| | c]quinolin-7- | 60/581,205 |
| | yl)phenyl]methanesulfonamide | Example 2 |

TABLE 2

| SEQ ID | CpG/type | Sequence* |
|---|---|---|
| SEQ ID NO: 1 | K23/B | 5′-TCGAGCGTTGTC-3′ |
| SEQ ID NO: 2 | 2216/A | 5′-GGgggacgatcgtcGGGGGg-3′ |
| SEQ ID NO: 3 | 1668/Murine | 5′-TCCATGACGTTCCTGATGCT-3′ |
| SEQ ID NO: 4 | 2006/B | 5′-TCGTCGTTTTGTCGTTTTGTCGT |
| | | T-3′ |
| SEQ ID NO: 5 | M352/C | 5′-TCGTCGAACGTTCGAGATGA |
| | | T-3′ |
| SEQ ID NO: 6 | 5192/B | 5′-TCGTCGTTTTTTTTTTT-3′ |
| SEQ ID NO; 7 | 2059/B | 5′-tcgtcgttttgtcgttttgtcgt |
| | | t-3′ |
| SEQ ID NO: 8 | | 5′-AAAAA-3′ |
| SEQ ID NO: 9 | | 5′-AAAAAAAAAAA-3′ |
| SEQ ID NO: 10 | | 5′-AAAAAAAAAAAAA-3′ |
| SEQ ID NO: 11 | | 5′-AAAAAAAAAAAAAAAAA-3′ |
| SEQ ID NO: 12 | | 5′-CCCCC-3′ |
| SEQ ID NO: 13 | | 5′-CCCCCCCCCC-3′ |
| SEQ ID NO: 14 | | 5′-CCCCCCCCCCCCCCC-3′ |
| SEQ ID NO: 15 | | 5′-CCCCCCCCCCCCCCCCCCCC-3′ |
| SEQ ID NO: 16 | | 5′-TTTTT-3′ |
| SEQ ID NO: 17 | | 5′-TTTTTTTT-3′ |
| SEQ ID NO: 18 | | 5′-TTTTTTTTTTT-3′ |
| SEQ ID NO: 19 | | 5′-TTTTTTTTTTTTTT-3′ |

* Upper case letters indicate a phosporothioate linkage 3′ of the base; lower case letters indicate a phosphodiester linkage 3′ of the base.

SEQ ID NO:1 is reported in Gürsel et al., J. Leukoc. Biol. (2002), vol. 71, pp. 813-820. SEQ ID NO:2, SEQ ID NO:4, and SEQ ID NO:5 are reported in Hartmenn et al., Eur. J. Immunol. (2003), vol. 33, pp. 1633-1641. SEQ ID NO:3 is reported in Zhu et al., J. Leukoc. Biol. (2002), vol. 72, pp, 1154-1163. SEQ ID NO:6 and SEQ ID NO:7 are reported in Vollmer et al., Antisense Nucleic Acid Drug Dev. (2002), vol. 12, pp. 165-175.

### Example 1

Human TLR8 and NF-κβ were transfected into human epithelial kidney 293 (HEK293, American Type Culture Collection, Manassas, Va., ATCC No. CRL-1573) cells as described in International Patent Publication Nos. WO2004/071459. The selected transfected cells were counted and resuspended to a concentration of 5×10⁵ cell per mL in culture media.

Cultured media was prepared from complete DMEM media (Biosource International Inc., Camarillo, Calif.), without phenol red. Fetal bovine serum (Biosource International Inc.) was added to a final concentration of 10% (vol/vol.), sodium pyruvate (Biosource International Inc.) was added to 1 mM; L-glutamine (Biosource International Inc.) was added to 2 mM; penicillin (Biosource International Inc.) was added to 100 U/mL; streptomycin (Biosource International Inc.) was added to 100 μg/mL.

100 μL aliquots of cells were placed in the wells of a white-walled, white-bottomed 96-well plate (Corning, Inc. Corning, N.Y.). Cell aliquots were treated by adding CpG ODN K23 (SEQ ID NO:1), CpG ODN 5192 (SEQ ID NO:6), or CPG ODN 2059 (SEQ ID NO:7) (Invitrogen Corp., Carlsbad, Calif.) at a concentration of 0.1 μM, 0.5 μM, 1.0 μM, 5.0 μM, 10 μM, or 50 μM to the culture with or without 3 μM of IRM1. As additional controls, some cell aliquots were incubated with 3 μM of IRM1 alone, while other cell aliquots were incubated without a stimulus (media control). In all cases, the cells were incubated overnight at 37° C. with 5% CO₂ and 98% humidity.

After the cells incubated overnight, 100 μL volume of reconstituted LucLight Plus (Packard Instruments, Meriden, Conn.) was added to each, aliquot of cells. Each well of the plate was read on a L-max luminometer (Molecular Devices, Sunnyvale, Calif.). The data is expressed as fold increase of luciferase induction in cell aliquots incubated with the indicated stimulant compared to the negative control. Results are shown in FIG. 1.

### Example 2

HEK 293 cells expressing human TLR8 were prepared as described in Example 1. Cell aliquots were treated by adding CpG ODN M352 (SEQ ID NO:5) (Invitrogen Corp., Carlsbad, Calif.) at a concentration of 1.0 μM, 3.0 μM, 10 μM, or 30 μM to the culture with or without 3 μM of IRM1. As controls, some cell aliquots were incubated with 3 μM of IRM1 and other cell aliquots were incubated without a stimulus (media control). In all cases, the cells were incubated overnight at 37° C. with 5% CO₂ and 98% humidity.

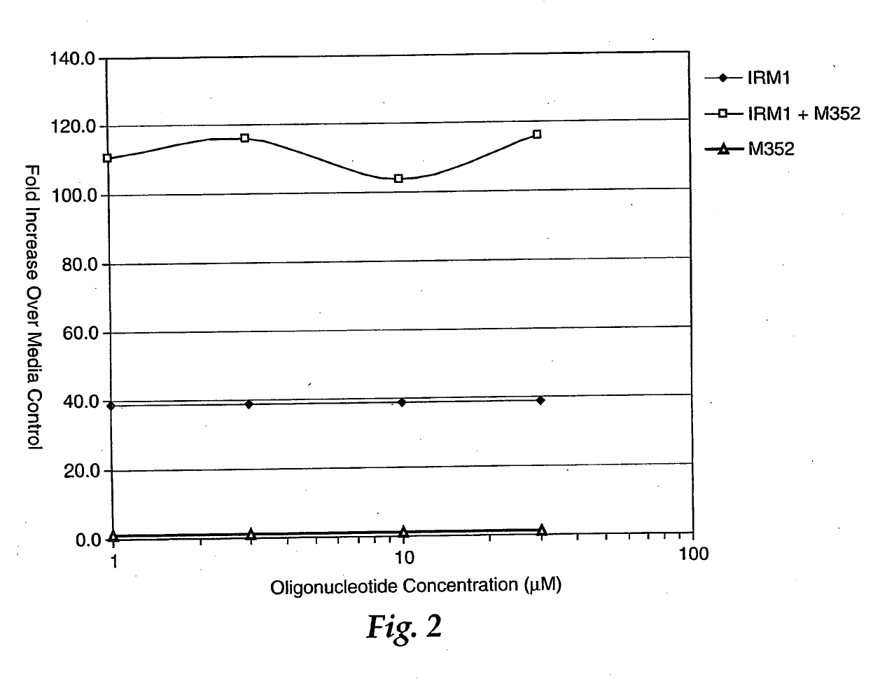

After the cells incubated overnight, 100 μL volume of reconstituted LucLight Plus (Packard Instruments, Meriden, Conn.) was added to each aliquot of cells. Each well of the plate was read on a L-max luminometer (Molecular Devices, Sunnyvale, Calif.). The data is expressed as fold increase of luciferase induction in cell aliquots incubated with the indicated stimulant compared to the negative control. Results are shown in FIG. 2.

### Example 3

Peripheral blood mononuclear cells (PBMCs) were enriched from human peripheral blood by HISTOPAQUE-1077 (Sigma-Aldrich Co., St. Louis, Mo.) density gradient centrifugation. PBMCs were counted and resuspended in complete RPMI 1640 with 25 mM HEPES (Biosource International Inc.) media. Fetal bovine serum (Biosource International Inc.) was added to a final concentration of 10% (vol/vol.), L-glutamine (Biosource International Inc.) was added to 2 mM; penicillin (Biosource International Inc.) was added to 100 U/mL; streptomycin (Biosource International Inc.) was added to 100 μg/mL.

5×10⁵ cells per well in 200 μL were placed in flat-bottom 96-well plate (Becton Dickenson Labware, Franklin Lakes, N.J.). Cell aliquots were treated by adding 10 μM of IRM2 alone (control) or with CpG ODN K23 (SEQ ID NO:1), CpG ODN 2006 (SEQ ID NO:4), or CpG ODN 2216 (SEQ ID NO:2) (Invitrogen Corp.) at a concentration of 0.03 μM, 0.1 μM, 0.3 μM, 1.0 μM, or 3.0 μM. In all cases, the cells were incubated overnight at 37° C. with 5% CO₂ and 98% humidity.

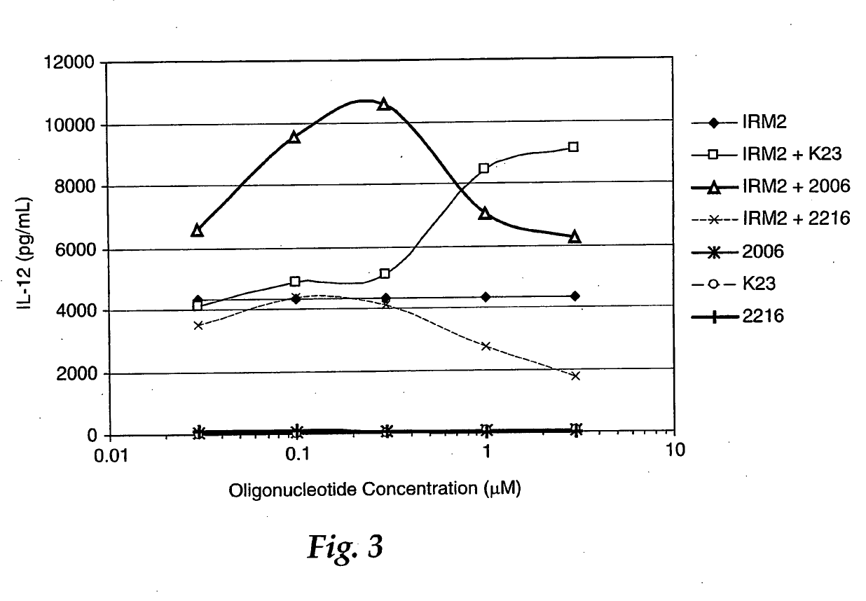

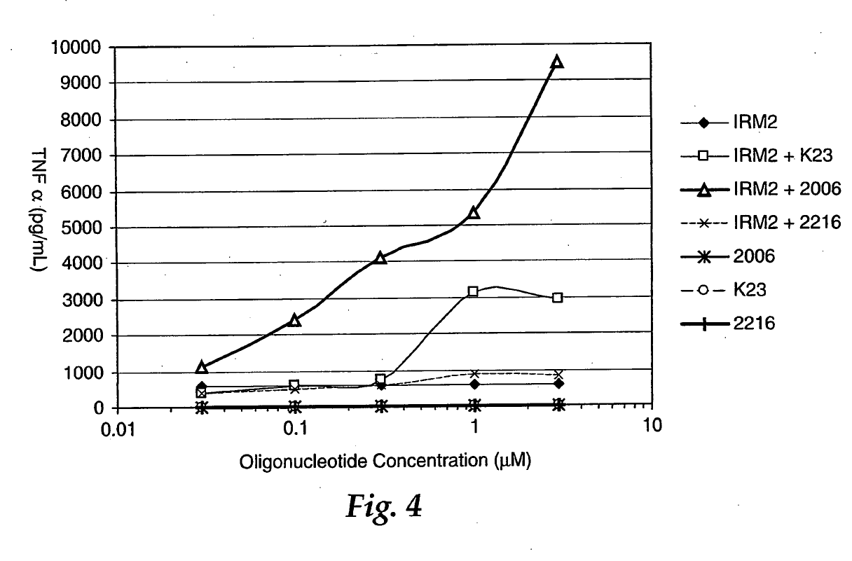

Culture supernatants were analyzed for IL-12 (pg/mL) or TNF (pg/mL) production using a human-specific IL-12 and TNF BV™ immunoassays (BioVeris Corp., Gaithersburg, Md.). Results are shown in FIG. 3 and FIG. 4.

### Example 4

Human monocyte-derived dendritic cells (mDCs) were generated as described in International Patent Publication No. WO2004/071459.

1×10⁵ cells per well in 200 μL were placed in flat-bottom 96-well plate (Becton Dickenson Labware, Franklin Lakes, N.J.). Cell aliquots were treated by adding 3 μM of IRM1 alone (control) or with CpG ODN K23 (SEQ ID NO:1), CpG ODN 2006 (SEQ ID NO:4), CpG ODN M352 (SEQ ID NO:5), or CpG ODN 2216 (SEQ ID NO:2) (Invitrogen Corp.) at a concentration of 0.03 μM, 0.1 μM, 0.3 μM, 1.0 μM, or 3.0 μM. In all cases, the cells were incubated overnight at 37° C. with 5% CO₂ and 98% humidity.

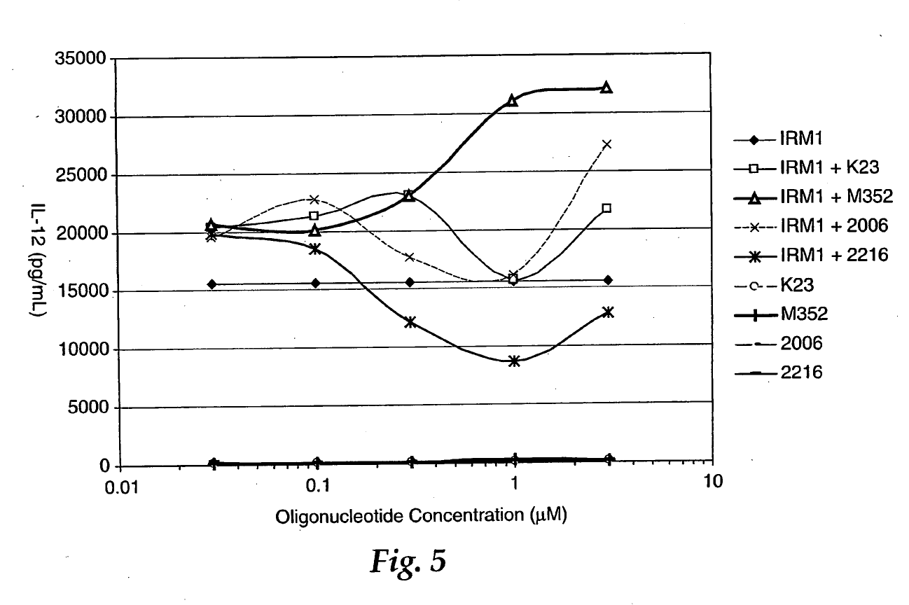

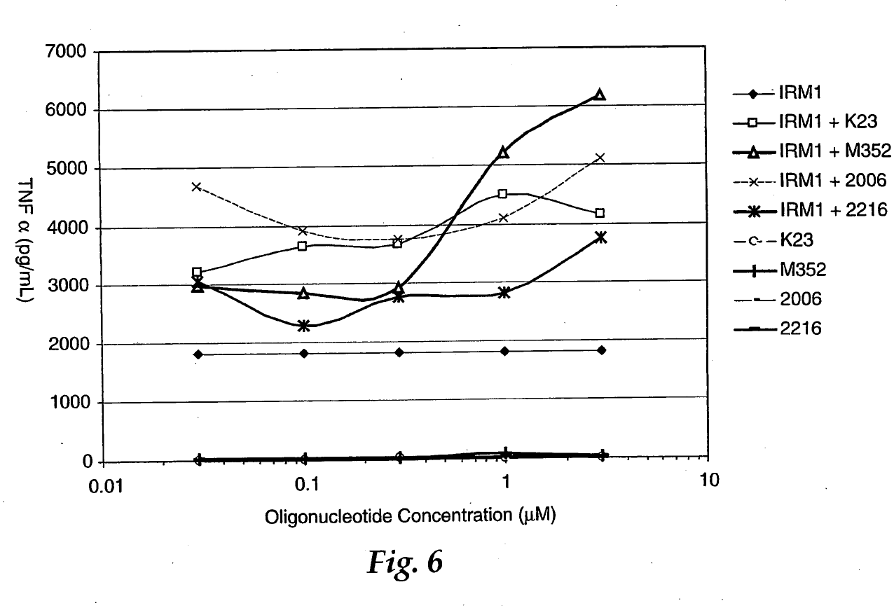

Culture supernatants were analyzed for IL-12 (pg/mL) or TNF (pg/mL) production using a human-specific IL-12 & TNF BV™ immunoassays (BioVeris Corp., Gaithersburg, Md.). Results are shown in FIG. 5 and FIG. 6.

### Example 5

HEK293 cells expressing human TLR8 were prepared as described in Example 1. Cell aliquots were treated with 1 μM of IRM1 alone (control) or with a 5-mer (SEQ ID NO:8), 11-mer (SEQ ID NO:9), 13-mer (SEQ ID NO:10), or 17-mer (SEQ ID NO:11) poly(A) oligonucleotide sequence (Invitrogen Corp.) at a concentration of 0.03 μM, 0.12 μM, 0.3 μM, 1.1 μM, 3.3 μM, 10 μM, 30 μM, or 100 μM. As a negative control, some cell aliquots were incubated without a stimulus (media control).

After the cells incubated overnight, the cells were analyzed for TNF production as described in Example 3. The data is expressed as fold increase of luciferase induction in cell aliquots incubated with the indicated stimulant compared to the negative control. Results are shown in FIG. 7.

### Example 6

PBMCs were prepared as described in Example 3. Cell aliquots were treated with 3 μM of IRM1 alone (control) or with a poly(C) oligonucleotide (5-mer, SEQ ID NO:12; 10-mer, SEQ ID NO:13; 15-mer, SEQ ID NO:14; or 25-mer, SEQ ID NO:15) or poly(T) oligonucleotide (5-mer, SEQ ID NO:16; 8-mer, SEQ ID NO:17; 11-mer, SEQ ID NO:18; or 14-mer, SEQ ID NO:19) (Invitrogen Corp.) at a concentration of 0.03 μM, 0.12 μM, 0.3 μM, 1.1 μM, 3.3 μM, 10 μM, or 30 μM.

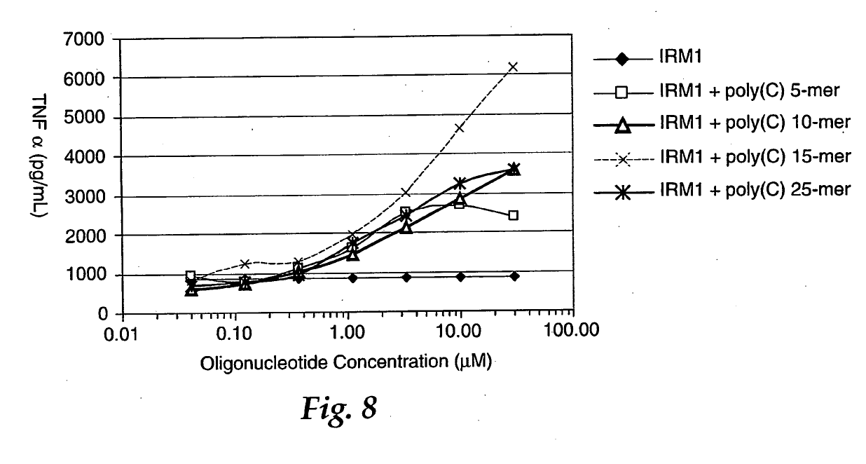

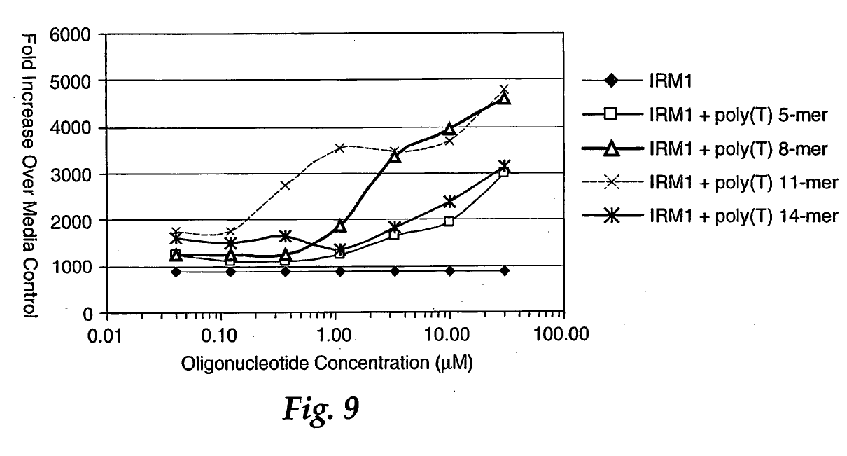

Culture supernatants were analyzed for TNF (pg/mL) production using a human-specific IL-12 and TNF BV™ immunoassays (BioVeris Corp., Gaithersburg, Md.). Results are shown in FIG. 8 and FIG. 9.

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

What is claimed is:

1. An immunostimulatory combination that comprises:
a TLR8 agonist in an amount that, in combination with an immunostimulatory oligonucleotide, is capable of inducing at least one TLR8-mediated biological activity; and
an immunostimulatory oligonucleotide in an amount effective to increase the extent to which the TLR8 agonist induces the at least one TLR8-mediated biological activity;
wherein the TLR8 agonist comprises a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

2. The immunostimulatory combination of claim 1 wherein the immunostimulatory oligonucleotide comprises a CpG oligodinucleotide.

3. The immunostimulatory combination of claim 2 wherein the CpG oligodinucleotide comprises a CpG-A oligodinucleotide.

4. The immunostimulatory combination of claim 2 wherein the CpG oligodinucleotide comprises a CpG-B oligodinucleotide.

5. The immunostimulatory combination of claim 2 wherein the CpG oligodinucleotide comprises a CpG-C oligodinucleotide.

6. The immunostimulatory combination of claim 1 wherein the immunostimulatory oligonucleotide comprises a poly(T) oligonucleotide, a poly(A) oligonucleotide, or poly(C) oligonucleotide.

7. The immunostimulatory combination of claim 1 wherein the TLR8 agonist and the immunostimulatory oligonucleotide are provided in a single formulation.

8. An immunostimulatory combination that comprises:
a TLR8 agonist in an amount that, in combination with an immunostimulatory oligonucleotide, is capable of inducing at least one TLR8-mediated biological activity; and
an immunostimulatory oligonucleotide in an amount effective to increase the extent to which the TLR8 agonist induces the at least one TLR8-mediated biological activity;
wherein the immunostimulatory oligonucleotide comprises an oligonucleotide that is other than a CpG oligonucleotide.

9. The immunostimulatory combination of claim 8 wherein the TLR8 agonist comprises an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

10. The immunostimulatory combination of claim 9 wherein the TLR8 agonist comprises an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

11. The immunostimulatory combination of claim 9 wherein the TLR8 agonist comprises a thiazoloquinoline amine.

12. The immunostimulatory combination of claim 8 wherein the TLR8 agonist and the immunostimulatory oligonucleotide are provided in a single formulation.

13. An immunostimulatory combination that comprises:
a TLR8 agonist in an amount that, in combination with an immunostimulatory oligonucleotide, is capable of inducing at least one TLR8-mediated biological activity; and
an immunostimulatory oligonucleotide in an amount effective to provide a synergistic increase in the at least one TLR8-mediated biological activity induced by the TLR8 agonist.

14. The immunostimulatory combination of claim 13 wherein the synergistic increase in TLR8-mediated biological activity is at least two-fold over that induced by the TLR8 agonist without the immunostimulatory oligonucleotide.

15. The immunostimulatory combination of claim 13 wherein the synergistic increase in TLR8-mediated biological activity is at least three-fold over that induced by the TLR8 agonist without the immunostimulatory oligonucleotide.

16. The immunostimulatory combination of claim 13 wherein the synergistic increase in TLR8-mediated biological activity is at least five-fold over that induced by the TLR8 agonist without the immunostimulatory oligonucleotide.

17. A method of inducing TLR8-mediated biological activity in immune cells, the method comprising:
contacting the immune cells with an immunostimulatory combination that comprises a TLR8 agonist and an immunostimulatory oligonucleotide in an amount effective to increase a TLR8-mediated biological activity of the cells to a greater extent than contacting the immune cells with the TLR8 agonist without the immunostimulatory oligonucleotide;
wherein the TLR8 agonist comprises an IRM compound that comprises a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

18. The method of claim 17 wherein the immunostimulatory oligonucleotide comprises a CpG oligodinucleotide.

19. The method of claim 18 wherein the CpG oligodinucleotide comprises a CpG-A.

20. The method of claim 18 wherein the CpG oligodinucleotide comprises a CpG-B oligodinucleotide.

21. The method of claim 18 wherein the CpG oligodinucleotide comprises a CpG-C oligodinucleotide.

22. The method of claim 17 wherein the immune cells comprise PBMCs or monocyte-derived dendritic cells.

23. The method of claim 17 wherein the TLR8-mediated biological activity comprises synthesis of a cytokine, synthesis of a chemokine, synthesis of co-stimulatory markers, maturation of antigen-presenting cells, or proliferation of B lymphocytes.

24. The method of claim 23 wherein the cytokine comprises TNF or IL-12.

25. The method of claim 17 wherein contacting the immune cells with an immunostimulatory combination comprises adding the immunostimulatory combination to isolated immune cells in vitro.

26. The method of claim 17 wherein contacting the immune cells with an immunostimulatory combination comprises administering the immunostimulatory combination to a subject in a manner that permits the immunostimulatory combination to contact immune cells of the subject in vivo.

27. The method of claim 17 wherein the immunostimulatory oligonucleotide comprises a poly(T) oligonucleotide, a poly(A) oligonucleotide, or a poly(C) oligonucleotide.

28. The method of claim 17 wherein the IRM compound and the immunostimulatory oligonucleotide form an IRM-immunostimulatory oligonucleotide complex.

29. The method of claim 28 wherein the IRM-immunostimulatory oligonucleotide complex includes intercalation of the IRM compound into the immunostimulatory oligonucleotide.

30. A method of inducing TLR8-mediated biological activity in immune cells, the method comprising:
contacting the immune cells with an immunostimulatory combination that comprises a TLR8 agonist and an immunostimulatory oligonucleotide in an amount effective to increase a TLR8-mediated biological activity of the cells to a greater extent than contacting the immune cells with the TLR8 agonist without the immunostimulatory oligonucleotide;
wherein the immunostimulatory oligonucleotide comprises an oligonucleotide other than a CpG oligonucleotide.

31. The method of claim 30 wherein the TLR8 agonist comprises an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

32. The method of claim 31 wherein the TLR8 agonist comprises an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

33. The method of claim 31 wherein the TLR8 agonist comprises a thiazoloquinoline amine.

34. The method of claim 30 wherein the TLR8 agonist and the immunostimulatory oligonucleotide form a complex in which the TLR8 agonist intercalates into the immunostimulatory oligonucleotide.

35. A method of inducing TLR8-mediated biological activity in immune cells, the method comprising:
contacting the immune cells with an immunostimulatory combination that comprises a TLR8 agonist and an immunostimulatory oligonucleotide in an amount effective to provide a synergistic increase in the at least one TLR8-mediated biological activity induced by the TLR8 agonist.

36. The method of claim 35 wherein the synergistic increase in TLR8-mediated biological activity is at least two-fold over that induced by the TLR8 agonist without the immunostimulatory oligonucleotide.

37. The method of claim 35 wherein the synergistic increase in TLR8-mediated biological activity is at least three-fold over that induced by the TLR8 agonist without the immunostimulatory oligonucleotide.

38. The method of claim 35 wherein the synergistic increase in TLR8-mediated biological activity is at least five-fold over that induced by the TLR8 agonist without the immunostimulatory oligonucleotide.

39. The method of claim 36 wherein the TLR8 agonist and the immunostimulatory oligonucleotide form a complex in which the TLR8 agonist intercalates into the immunostimulatory oligonucleotide.


SEQUENCE LISTING (19 sequences)

SEQ ID NO: 1 - DNA, 12 bp, Artificial sequence
  Synthetic polynucleotide
tcgagcgttgtc

SEQ ID NO: 2 - DNA, 20 bp, Artificial sequence
  Synthetic polynucleotide
gggggacgatcgtcgggggg

SEQ ID NO: 3 - DNA, 20 bp, Artificial sequence
  Synthetic polynucleotide
tccatgacgttcctgatgct

SEQ ID NO: 4 - DNA, 24 bp, Artificial sequence
  Synthetic polynucleotide
tcgtcgttttgtcgttttgtcgtt

SEQ ID NO: 5 - DNA, 21 bp, Artificial sequence
  Synthetic polynucleotide
tcgtcgaacgttcgagatgat

SEQ ID NO: 6 - DNA, 17 bp, Artificial sequence
  Synthetic polynucleotide
tcgtcgttttttttttt

SEQ ID NO: 7 - DNA, 24 bp, Artificial sequence
  Synthetic polynucleotide
tcgtcgttttgtcgttttgtcgtt

SEQ ID NO: 8 - DNA, 5 bp, Artificial sequence
  Synthetic polynucleotide
aaaaa

SEQ ID NO: 9 - DNA, 11 bp, Artificial sequence
  Synthetic polynucleotide
aaaaaaaaaaa

SEQ ID NO: 10 - DNA, 13 bp, Artificial sequence
  Synthetic polynucleotide
aaaaaaaaaaaaa

SEQ ID NO: 11 - DNA, 17 bp, Artificial sequence
  Synthetic polynucleotide
aaaaaaaaaaaaaaaaa

SEQ ID NO: 12 - DNA, 5 bp, Artificial sequence
  Synthetic polynucleotide
ccccc

SEQ ID NO: 13 - DNA, 10 bp, Artificial sequence
  Synthetic polynucleotide
cccccccccc

SEQ ID NO: 14 - DNA, 15 bp, Artificial sequence
  Synthetic polynucleotide
ccccccccccccccc

SEQ ID NO: 15 - DNA, 20 bp, Artificial sequence
  Synthetic polynucleotide
cccccccccccccccccccc

SEQ ID NO: 16 - DNA, 5 bp, Artificial sequence
  Synthetic polynucleotide
ttttt

SEQ ID NO: 17 - DNA, 8 bp, Artificial sequence
  Synthetic polynucleotide
tttttttt

SEQ ID NO: 18 - DNA, 11 bp, Artificial sequence
  Synthetic polynucleotide
ttttttttttt

SEQ ID NO: 19 - DNA, 14 bp, Artificial sequence
  Synthetic polynucleotide
tttttttttttttt